(19) United States
(12) Patent Application Publication
(10) Pub. No.: US 2024/0043758 A1
(43) Pub. Date: Feb. 8, 2024

(54) Process for Removing Benzene from a Heart-Cut Reformate

(71) Applicants: Phillips 66 Company, Houston, TX (US); SINOPEC Shanghai Research Institute of Petrochemical Technology Co., Ltd., Shanghai (CN)

(72) Inventors: Jianhua Yao, Bartlesville, OK (US); Dhananjay Ghonasgi, Bartlesville, OK (US); Kening Gong, Bartlesville, OK (US); Sourabh Pansare, Bartlesville, OK (US); Weimin Yang, Shanghai (CN); Huanxin Gao, Shanghai (CN); Wennian Wang, Shanghai (CN); Ming Xu, Shanghai (CN); Yilun Wei, Shanghai (CN)

(21) Appl. No.: 18/365,938

(22) Filed: Aug. 4, 2023

Related U.S. Application Data
(60) Provisional application No. 63/395,052, filed on Aug. 4, 2022.

(51) Int. Cl. C10G 49/08; C07C 2/76; B01J 23/46; B01J 23/44; B01J 23/42; B01J 29/06; B01J 23/89; B01J 23/83; B01J 21/06

(52) U.S. Cl. CPC C10G 49/08; C07C 2/76; B01J 23/46; B01J 23/44; B01J 23/42; B01J 29/06; B01J 23/892; B01J 23/8906; B01J 23/8926; B01J 23/83; B01J 21/066; C10G 2400/04; C10G 2300/1096; C10G 2300/307; C10G 2300/308

(57) ABSTRACT

The disclosed process relates to removal of benzene from a reformate stream and in turn providing gasoline and diesel products along with commodity chemicals (such as cyclohexylbenzene). The disclosed process further relates to the upgrading of heart-cut reformate benzene to higher value products.

### INCORPORATION BY REFERENCE

The entire content of each document listed below is incorporated by reference into this document (the documents below are collectively referred to as the “incorporated documents”). If the same term is used in both this document and one or more of the incorporated documents, then it should be interpreted to have the broadest meaning imparted by any one or combination of these sources unless the term has been explicitly defined to have a different meaning in this document. If there is an inconsistency between any incorporated document and this document, then this document shall govern. The incorporated subject matter should not be used to limit or narrow the scope of the explicitly recited or depicted subject matter.

Priority patent documents incorporated by reference:
U.S. Prov. App. No. 63/395,052, titled “Process for Removing Benzene from a Heart-Cut Reformate,” filed on 4 Aug 2023.

### FIELD

The disclosed process relates to removal of benzene from a reformate stream and in turn providing gasoline and diesel products along with commodity chemicals (such as cyclohexylbenzene). The disclosed process further relates to the upgrading of heart-cut reformate benzene to higher value products.

### BRIEF DESCRIPTION OF THE FIGURES

FIG. 1 outlines the use of the catalytic unit operation in process 100.

FIGS. 2A-2C outline the various aspects of general process 200 (and variations on process 200 including 220 and 230) wherein the catalytic unit operation is configured to deliver the product stream to a separation unit.

FIG. 3 outlines process 300 wherein the catalytic unit operation is configured to receive a heart-cut reformate from a splitter.

FIG. 4 outlines the disclosed process 400 configured to produce naphtha, commodity chemicals, and diesel.

### DETAILED DESCRIPTION

The materials, compounds, compositions, articles, and methods described herein may be understood more readily by reference to the following detailed description of specific aspects of the disclosed subject matter and the Examples included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings:

All percentages, ratios and proportions herein are by weight, unless otherwise specified. All temperatures are in degrees Celsius (° C.) unless otherwise specified.

The terms “a” and “an” are defined as one or more unless this disclosure explicitly requires otherwise.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Values expressed as “greater than” do not include the lower value. For example, when the “variable x” is defined as “greater than zero” expressed as “0<x” the value of x is any value, fractional or otherwise that is greater than zero.

Similarly, values expressed as “less than” do not include the upper value. For example, when the “variable x” is defined as “less than 2” expressed as “x<2” the value of x is any value, fractional or otherwise that is less than 2.

“Optional” or “optionally” means that the subsequently described event or circumstance can or cannot occur, and that the description includes instances where the event or circumstance occurs and instances where it does not.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, an apparatus that “comprises,” “has,” “includes” or “contains” one or more elements possesses those one or more elements but is not limited to possessing only those elements Likewise, a method that “comprises,” “has,” “includes” or “contains” one or more steps possesses those one or more steps but is not limited to possessing only those one or more steps.

Any embodiment of any of the apparatuses, systems, and methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, to change the scope of a given claim from what it would otherwise be using the open-ended linking verb.

The feature or features of one embodiment may be applied to other embodiments, even though not described or illustrated, unless expressly prohibited by this disclosure or the nature of the embodiments.

As used herein the term “unit operation” is defined as a distinct chemical transformation step. Non-limiting examples of unit operations includes chemical reaction, isolation, separation, evaporation, filtration, admixing of two or more substances. The disclosed unit operations can be combined with other unit operations to define, for example, an overall petroleum refining or product forming process.

As used herein the terms “diesel,” “diesel cut” and “diesel fraction” are defined as products obtained by the disclosed process having a boiling point in the range of 300° F. to 750° F. and including n-paraffinic, iso-paraffinic, naphthenic, and aromatic hydrocarbons, ranging approximately from C₁₀ to C₂₀.

As used herein the term “pre-diesel fraction” is defined as a product stream that can be further treated, for example, in a hydrotreater to form a high cetane diesel fraction.

As used herein the term “high cetane” is defined as a product stream comprising at least about 50% by weight of saturated hydrocarbons and having the cetane number about 40 to about 70. The ASTM D613 is the method used to determine the level of cetane.

As used herein the term “cetane blend stock” is defined herein as diesel range hydrocarbon streams which have a cetane number of about 40 to 70 and which can be blended to form commercial diesel fuel. The ASTM D613 is the method used to determine the level of cetane.

As used herein the term “commodity fraction” or “commodity chemical” is used interchangeably throughout the Specification and is defined herein as a stream which can be used as a chemical reagent.

As used herein the term “naphthenes” is defined as alkyl substituted or unsubstituted hydrocarbon rings having the formula CₙH₂ₙ wherein the index n is about 5 to about 8. Cyclohexane, methylcyclopropane, and methylcyclohexane are examples of naphthenes. In use naphthenes are a source of gasoline blending stock.

The present disclosure relates to petroleum oil separation and feed stream upgrading. In addition, the disclosed process provides petroleum blending stocks having low benzene content.

In one aspect, disclosed herein is a catalytic unit operation, comprising introducing hydrogen gas into an incoming heart-cut reformate stream, further directing the stream into a catalytic reaction zone comprising a catalyst and heating the stream in the presence of the catalyst in the catalyst reaction zone to form a product stream.

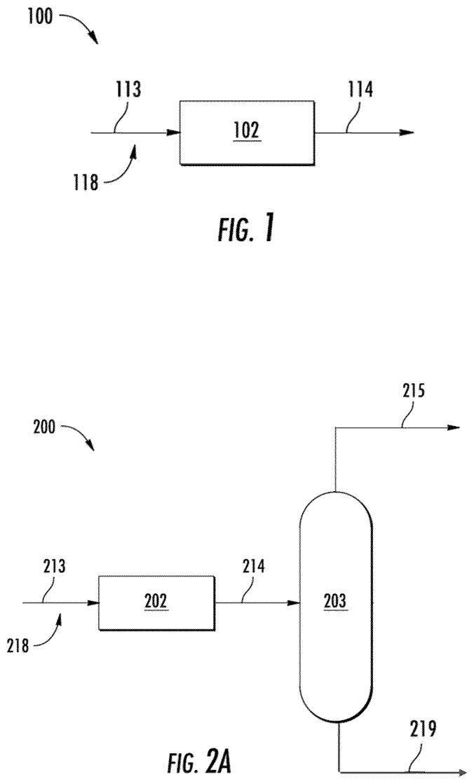

This aspect is exemplified in FIG. 1. Process 100 comprises the steps wherein a heart-cut reformate stream 113 is received from a heart-cut reformate source, the stream is combined with a source of hydrogen gas via feed line 118 and the stream/hydrogen admixture is further directed into a catalytic reaction zone 102 comprising one or more of the disclosed hydroalkylation catalysts. Once in the catalytic reaction zone 102 the heart-cut reformate/hydrogen gas admixture is heated inside the catalyst reaction zone unit operation 102 thereby producing the hydroalkylated heart-cut reformat which exits the catalyst reaction unit operation 102 via product stream 114.

In one embodiment the heart-cut reformate stream comprises about 5% to about 75% by weight of benzene and the catalytic unit operation 102 converts at least 40% of the benzene in the heart-cut reformate stream to non-benzene hydrocarbons. In another embodiment the heart-cut reformate stream comprises about 25% to about 75% by weight of benzene. In a yet another embodiment the heart-cut reformate stream comprises about 50% to about 75% by weight of benzene. In a still yet another embodiment the heart-cut reformate stream comprises about 20% to about 50% by weight of benzene.

The disclosed heart-cut reformate streams can comprise about 5% to about 75% by weight of benzene, for example, 5%, 6%, 7%, 8%, 9%, 10%, 11%, 12%, 13%, 14%, 15%, 16%, 17%, 18%, 19%, 20%, 21%, 22%, 23%, 24%, 25%. 26%, 27, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, or 75%.

In one iteration of this embodiment the catalytic unit operation 102 converts about 40% to about 99% of the benzene in the heart-cut reformate stream to non-benzene hydrocarbons. In another iteration of this embodiment the catalytic unit operation converts about 50% to about 95% of the benzene in the heart-cut reformate stream to non-benzene hydrocarbons. In a further iteration of this embodiment the catalytic unit operation converts about 40% to about 95% of the benzene in the heart-cut reformate stream to non-benzene hydrocarbons. In a yet further iteration of this embodiment the catalytic unit operation converts about 50% to about 85% of the benzene in the heart-cut reformate stream to non-benzene hydrocarbons. In another further iteration of this embodiment the catalytic unit operation converts about 75% to about 99% of the benzene in the heart-cut reformate stream to non-benzene hydrocarbons.

The disclosed catalytic unit operation can convert about 40% to about 99% of the benzene in the heart-cut reformate stream to non-benzene hydrocarbons, for example,

40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 90%, 91%, 92%, 93%, 94%, 95%, 96%, 97%, 98%, or 99%.

In one embodiment the disclosed catalytic reaction zone in a catalytic unit operation can be heated to a temperature of about 100° C. to about 250° C. In one embodiment the disclosed catalytic reaction zone in a catalytic unit operation can be heated to a temperature of about 140° C. to about 200° C. In one embodiment the disclosed catalytic reaction zone in a catalytic unit operation can be heated to a temperature of about 170° C. to about 200° C. In one embodiment the disclosed catalytic unit operation catalytic reaction zone can be heated to a temperature of about 150° C. to about 180° C. In one embodiment the disclosed catalytic reaction zone in a catalytic unit operation can be heated to a temperature of about 150° C. to about 170° C.

The disclosed catalytic unit operation in catalytic reaction zone can be heated to a temperature of about 100° C. to about 250° C., for example, 100° C., 101° C., 102° C., 103° C., 104° C., 105° C., 106° C., 107° C., 108° C., 109° C., 110° C., 111° C., 112° C., 113° C., 114° C., 115° C., 116° C., 117° C., 118° C., 119° C., 120° C., 121° C., 122° C., 123° C., 124° C., 125° C., 126° C., 127° C., 128° C., 129° C., 130° C., 131° C., 132° C., 133° C., 134° C., 135° C., 136° C., 137° C., 138° C., 139° C., 140° C., 141° C., 142° C., 143° C., 144° C., 145° C., 146° C., 147° C., 148° C., 149° C., 150° C., 151° C., 152° C., 153° C., 154° C., 155° C., 156° C., 157° C., 158° C., 159° C., 160° C., 161° C., 162° C., 163° C., 164° C., 165° C., 166° C., 167° C., 168° C., 169° C., 170° C., 171° C., 172° C., 173° C., 174° C., 175° C., 176° C., 177° C., 178° C., 179° C., 180° C., 181° C., 182° C., 183° C., 184° C., 185° C., 186° C., 187° C., 188° C., 189° C., 190° C., 191° C., 192° C., 193° C., 194° C., 195° C., 196° C., 197° C., 198° C., 199° C., 200° C., 201° C., 202° C., 203° C., 204° C., 205° C., 206° C., 207° C., 208° C., 209° C., 210° C., 211° C., 212° C., 213° C., 214° C., 215° C., 216° C., 217° C., 218° C., 219° C., 220° C., 221° C., 222° C., 223° C., 224° C., 225° C., 226° C., 227° C., 228° C., 229° C., 230° C., 231° C., 232° C., 233° C., 234° C., 235° C., 236° C., 237° C., 238° C., 239° C., 240° C., 241° C., 242° C., 243° C., 244° C., 245° C., 246° C., 247° C., 248° C., 249° C. and 250° C.

As shown in FIG. 1 heart-cut reformate stream 113 is admixed with hydrogen gas 118 before entering the catalytic reaction zone 102. In one embodiment the hydrogen gas is introduced into the unit operation heart-cut reformate stream at a pressure of about 50 psig to about 500 psig. In another embodiment the hydrogen gas is introduced into the unit operation heart-cut reformate stream at a pressure of about 50 psig to about 300 psig. In a further embodiment the hydrogen gas is introduced into the unit operation heart-cut reformate stream at a pressure of about 75 psig to about 200 psig. In a still further embodiment, the hydrogen gas is introduced into the unit operation heart-cut reformate stream at a pressure of about 100 psig to about 200 psig. In a yet further embodiment, the hydrogen gas is introduced into the unit operation heart-cut reformate stream at a pressure of about 125 psig to about 170 psig. In still another embodiment the hydrogen gas is introduced into the unit operation heart-cut reformate stream at a pressure of about 140 psig to about 160 psig.

The hydrogen gas pressure forming an admixture with the heart-cut reformate stream is about 50 psig to about 500 psig, for example, 50 psig, 51 psig, 52 psig, 53 psig, 54 psig, 55 psig, 56 psig, 57 psig, 58 psig, 59 psig, 60 psig, 61 psig, 62 psig, 63 psig, 64 psig, 65 psig, 66 psig, 67 psig, 68 psig, 69 psig, 70 psig, 71 psig, 72 psig, 73 psig, 74 psig, 75 psig, 76 psig, 77 psig, 78 psig, 79 psig, 80 psig, 81 psig, 82 psig, 83 psig, 84 psig, 85 psig, 86 psig, 87 psig, 88 psig, 89 psig, 90 psig, 90 psig, 91 psig, 92 psig, 93 psig, 94 psig, 95 psig, 96 psig, 97 psig, 98 psig, 99 psig, 100 psig, 101 psig, 102, psig, 103, psig, 104 psig, 105 psig, 106 psig, 107 psig, 108 psig, 109 psig, 110 psig, 111 psig, 112 psig, 113 psig, 114 psig, 115 psig, 116 psig, 117 psig, 118 psig, 119 psig, 120 psig, 121 psig, 122 psig, 123 psig, 124 psig, 125 psig, 126 psig, 127 psig, 128 psig, 129 psig, 130 psig 31 psig, 132 psig, 133 psig, 134 psig, 135 psig, 136 psig, 137 psig, 138 psig, 139 psig, 140 psig, 141 psig, 142 psig, 143 psig, 144 psig, 145 psig, 146 psig, 147 psig, 148 psig, 149 psig, 150 psig, 151 psig, 152 psig, 153 psig, 154 psig, 155 psig, 156 psig, 157 psig, 158 psig, 159 psig, 160 psig, 161 psig, 1 62 psig, 163 psig, 164 psig, 165 psig, 166 psig, 167 psig, 168 psig, 169 psig, 170 psig, 171 psig, 172 psig, 173 psig, 174 psig, 175 psig, 176 psig, 177 psig, 178 psig, 179 psig, 180 psig, 181 psig, 182 psig, 183 psig, 184 psig, 185 psig, 186 psig, 187 psig, 188 psig, 189 psig, 190 psig, 190 psig, 191 psig, 192 psig, 193 psig, 194 psig, 195 psig, 196 psig, 197 psig, 198 psig, 199 psig, 200 psig, 201 psig, 202, psig, 203, psig, 204 psig, 205 psig, 206 psig, 207 psig, 208 psig, 209 psig, 210 psig, 212 psig, 212 psig, 213 psig, 214 psig, 215 psig, 216 psig, 217 psig, 218 psig, 219 psig, 220 psig, 221 psig, 222 psig, 223 psig, 224 psig, 225 psig, 226 psig, 227 psig, 228 psig, 229 psig, 230 psig, 231 psig, 232 psig, 233 psig, 234 psig, 235 psig, 236 psig, 237 psig, 238 psig, 239 psig, 240 psig, 241 psig, 242 psig, 243 psig, 244 psig, 245 psig, 246 psig, 247 psig, 248 psig, 249 psig, 250 psig, 251 psig, 252 psig, 253 psig, 254 psig, 255 psig, 256 psig, 257 psig, 258 psig, 259 psig, 260 psig, 261 psig, 2 62 psig, 263 psig, 264 psig, 265 psig, 266 psig, 267 psig, 268 psig, 269 psig, 270 psig, 271 psig, 272 psig, 273 psig, 274 psig, 275 psig, 276 psig, 277 psig, 278 psig, 279 psig, 280 psig, 281 psig, 282 psig, 283 psig, 284 psig, 285 psig, 286 psig, 287 psig, 288 psig, 289 psig, 290 psig, 290 psig, 291 psig, 292 psig, 293 psig, 294 psig, 295 psig, 296 psig, 297 psig, 298 psig, 299 psig, 300 psig, 301 psig, 302, psig, 303, psig, 304 psig, 305 psig, 306 psig, 307 psig, 308 psig, 309 psig, 310 psig, 311psig 312 psig, 313 psig, 314 psig, 315 psig, 316 psig, 317 psig, 318 psig, 319 psig, 320 psig, 321 psig, 322 psig, 323 psig, 324 psig, 325 psig, 326 psig, 327 psig, 328 psig, 329 psig, 330 psig, 331 psig, 332 psig, 333 psig, 334 psig, 335 psig, 336 psig, 337 psig, 338 psig, 339 psig, 340 psig, 341 psig, 342 psig, 343 psig, 344 psig, 345 psig, 346 psig, 347 psig, 348 psig, 349 psig, 350 psig, 351 psig, 352 psig, 353 psig, 354 psig, 355 psig, 356 psig, 357 psig, 358 psig, 359 psig, 360 psig, 361 psig, 362 psig, 363 psig, 364 psig, 365 psig, 366 psig, 367 psig, 368 psig, 369 psig, 370 psig, 371 psig, 372 psig, 373 psig, 374 psig, 375 psig, 376 psig, 377 psig, 378 psig, 379 psig, 380 psig, 381 psig, 382 psig, 383 psig, 384 psig, 385 psig, 386 psig, 387 psig, 388 psig, 389 psig, 390 psig, 390 psig, 391 psig, 392 psig, 393 psig, 394 psig, 395 psig, 396 psig, 397 psig, 398 psig, 399 psig, 400 psig, 401 psig, 402, psig, 403, psig, 404 psig, 405 psig, 406 psig, 407 psig, 408 psig, 409 psig, 410 psig, 411 psig, 412 psig, 413 psig, 414 psig, 415 psig, 416 psig, 417 psig, 418 psig, 419 psig, 420 psig, 421 psig, 424 psig, 423 psig, 424 psig, 425 psig, 426 psig, 427 psig, 428 psig, 429 psig, 430 psig, 431 psig, 432 psig, 433 psig, 434 psig, 435 psig, 436 psig, 437 psig, 438 psig, 439 psig, 440 psig, 441 psig, 442 psig, 443 psig, 444 psig, 445 psig, 446 psig, 447 psig, 448 psig, 449 psig, 450 psig, 451 psig, 452 psig, 453 psig, 454 psig, 455 psig, 456 psig, 457 psig, 458 psig, 459 psig, 460 psig, 461 psig, 462 psig, 463 psig, 464 psig, 465 psig, 466 psig, 467 psig, 468 psig, 469 psig, 470 psig, 471 psig, 472 psig, 473 psig, 474 psig, 475 psig, 476 psig, 477 psig, 478 psig, 479 psig, 480 psig, 481 psig, 482 psig, 483 psig, 484 psig, 485 psig, 486 psig, 487 psig, 488 psig, 489 psig, 490 psig, 490 psig, 491 psig, 492 psig, 493 psig, 494 psig, 495 psig, 496 psig, 497 psig, 498 psig, 499 psig, or 500 psig.

In one embodiment the molar ratio of hydrogen gas to the amount of benzene in the heart-cut reformate stream is about 0.3:1 to about 4:1 molar ratio. For example, the molar ratio of hydrogen to benzene can be 0.3:1, 0.4:1, 0.5:1, 0.6:1, 0.7:1, 0.8:1, 0.9:1, 1:1, 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, or 4:1.

In one iteration of Process 100, stream 113 can be an enriched benzene, toluene, xylene isomer admixture (BTX) wherein the BTX admixture can be further processed to produce a commodity chemical such as cyclohexylbenzene.

Table I below shows the results of the benzene conversion and product distribution for experimental runs 1 to 4.

TABLE I

| Experiment No. | 1 | 2 | 3 | 4 |
|---|---|---|---|---|
| Pressure (psig) | 150 | 150 | 150 | 150 |
| Temperature (° C.) | 153 | 154 | 165 | 160 |
| Weight hourly space velocity | 1.6 | 1.6 | 1.6 | 1.6 |
| (hr⁻¹) | | | | |
| Hydrogen/Benzene molar | 1.1 | 1.6 | 2.0 | 2.0 |
| ratio | | | | |
| Percent benzene | 53.8 | 71.4 | 93.6 | 97.6 |
| conversion, % | | | | |

Product selectivity, wt %

| | | | | |
|---|---|---|---|---|
| Cyclohexane/C₆ alkanes | 27.7 | 27.9 | 55.9 | 43.3 |
| Cyclohexylbenzene (CHB) | 53.8 | 18.1 | 22.5 | 25.3 |
| Bicyclohexyl (BCH) | 0.3 | 0.4 | 0.4 | 1.1 |
| Other 2-ring compounds | 2.4 | 2.8 | 2.0 | 2.6 |
| 3-ring compounds | 15.8 | 20.8 | 19.1 | 27.8 |
| CHB purity via GC (%) | 99.3 | 98.9 | 97.8 | 95.1 |

As depicted in Table I the process yielded a high percentage of cyclohexylbenzene versus other ring products. Table I also indicates high cyclohexylbenzene purity that was estimated based on gas chromograph analysis for the stream within approximately ±10° C. of cyclohexylbenzene's boiling point.

In another aspect of the disclosed process the catalytic reaction zone unit operation can be in fluid communication with a separation unit 203 capable of separating stream 214 into various fractions that are formed in the catalytic reaction zone 202.

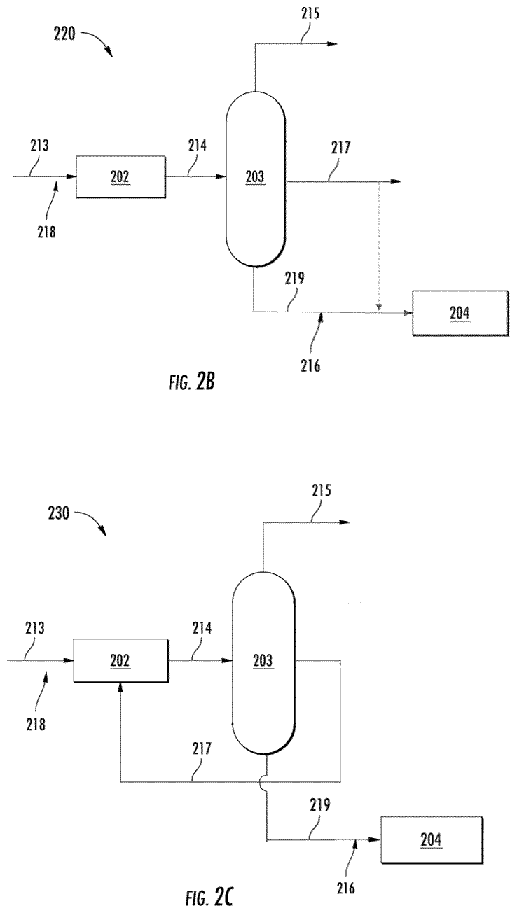

Process 200 depicts a catalytic reaction zone 202 in fluid connection with a separation unit operation 203. As shown in FIG. 2A a product stream 214 from the catalytic reaction zone 202 is fed into separation unit operation 203 where the product stream can be fractionated into product stream 215 comprising naphthenes, for example, cyclohexane and other alkyl substituted or unsubstituted hydrocarbon rings having the formula CₙH₂ₙ wherein the index n is about 6 to about 8. Product stream 215 can be directed to a gasoline pool or to another unit operation (not shown) for further processing. Bottoms outlet stream 219 can be directed to another unit operation (not shown) for further processing.

FIG. 2B depicts process 220 which is an iteration of process 200. In process 220 a pre-diesel stream 219 produced by separation unit 203 is in fluid connection with hydrotreater 204 where the pre-diesel stream 219 is converted to a high cetane diesel fraction. FIG. 2B further discloses an embodiment wherein a commodity stream 217 can also be produced from the separation unit 203. This commodity stream 217 contains a high concentration of cyclohexylbenzene that can be marketed separately or optionally be blended to pre-diesel product stream 219. The combined stream of 217 and 219 can be directed to a hydrotreating unit 204 for conversion to high cetane diesel. Stream 216 provides hydrogen that is necessary for the hydrotreating process.

FIG. 2C depicts process 230 which is an iteration of process 210 and 220. Unlike process 220, process 230 provides for recycling of a commodity stream 217 back to catalytic reaction zone 202 for further processing.

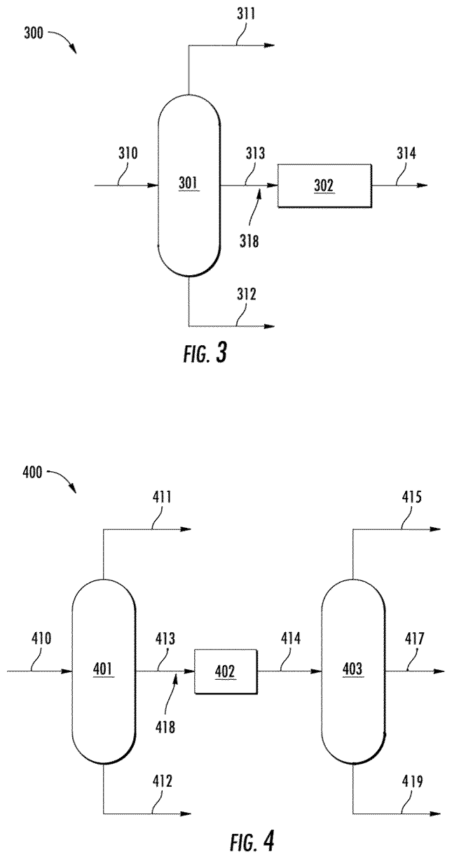

Process 300 is shown in FIG. 3 wherein a catalytic reaction zone unit operation 302 is in fluid connection via stream 313 with reformate splitter unit operation 301. The reformate splitter unit operation 301 receives via stream 310 a reformate admixture from a catalytic reformer or other sources. The stream 310, however, can be pretreated after exiting a catalytic reformer. The reformate splitter unit operation can separate out the heart-cut reformate via stream 313 from light reformate which exits via stream 311 and heavy reformate that exits via stream 312. Both the light reformate and heavy reformate can be returned to the gasoline pool.

Process 400 is shown in FIG. 4 which is a general process combining the elements of Process 100, Process 200, and Process 300. In use, stream 410 is received from a catalytic reformer or other source and directed to reformate splitter unit operation 401 where the reformer feed is split into a light reformate fraction exiting via stream 411, a heavy reformate fraction exiting via stream 412 and a heart-cut reformate fraction 413. The heart-cut reformate exits the reformate splitter unit operation 401 via stream 413 where it is admixed with hydrogen gas and directed to a catalytic reaction zone unit operation 402. With controlled desirable feed rates, the hydroalkylated product exits the catalytic reaction zone unit operation via stream 414 into a separation unit operation. The separation unit operation fractionates the incoming reaction zone product into various products, for example, stream 415 comprising naphthenes, for example, cyclohexane and C₆-C₈ alkyl hydrocarbon isomers having the formula CₙH₂ₙ wherein the index n is about 6 to about 8, stream 419 containing heavy aromatic pre-diesel compounds and stream 417 which contains a high concentration of cyclohexylbenzene. Stream 419 is optionally further directed to a hydrotreatment unit operation for conversion to high cetane diesel.

Disclosed herein is a catalytic hydroalkylation process for co-producing a high cetane diesel stream and a commodity chemical stream, comprising:
a) hydroalkylating a heart-cut reformate stream in the presence of a bifunctional hydroalkylation catalyst to form a treated reformate admixture; and
b) separating the components of the treated reformate admixture into various components;
wherein the components of the admixture are:
i) a first stream containing cyclohexane and C₆-C₈ hydrocarbon isomers; and
ii) a second stream containing cyclohexylbenzene, bicyclohexyl and other cyclic hydrocarbons; and
iii) a third stream containing dicyclohexylbenzene and dicyclohexylcyclo-hexane and other isomers.

In one iteration of this aspect of the disclosed process, the second stream is isolated as a commodity chemical product of cyclohexylbenzene.

Further disclosed herein is a catalytic hydroalylation process for preparing high cetane diesel stream, comprising:
a) hydroalkylating a heart-cut reformate stream in the presence of a bifunctional hydroalkylation catalyst to form a treated reformate admixture; and
b) separating the components of the treated reformate admixture into various components;
wherein the components of the admixture are:
i) a first stream containing cyclohexane and Cₙ naphthenes wherein the index n is from 6 to 8; and
ii) a second stream comprising cyclohexylbenzene, bicyclohexyl, dicyclohexylbenzene, dicyclohexylcyclohexane and their isomers.

In one iteration of this aspect of the disclosed process, the second stream is directed to a hydrotreating unit wherein any remaining cyclohexylbenzene, dicyclohexylbenzene and aromatic isomers are converted to bicyclohexyl, dicyclohexylcyclohexane and other saturated isomers wherein the resulting product can be used as high cetane diesel fuel.

The disclosed unit operation can be adapted to be in fluid connection with one or more unit operations. For example, the unit operation can be in adapted to deliver product stream or receive incoming streams, for example, a heart-cut stream.

### Catalyst

The disclosed catalysts are zeolite supported active metal catalysts capable of hydroalkylating a reformate stream to product a number of desired products, inter alia, a naphthene stream, a commodity chemical stream and an aromatic bottoms stream.

The stoichiometry of the reaction to form the zeolite support is based on the inorganic SiO₂ source. One aspect of the disclosed zeolites includes an inorganic SiO₂ to Al₂O₃ ratio of about 10 to about 100, an organic SiO₂ to Al₂O₃ ratio of about 0.005 to about 0.5, an inorganic base (OH⁻) to SiO₂ ratio of about 0.005 to about 1.0, water to SiO₂ ratio of about 10 to about 80, and an organic amine to SiO₂ ratio of about 0.05 to about 1.0.

The disclosed crystallization reaction temperature is about 100° C. to about 180° C., whereas the crystallization reaction time is about 2 hours to about 60 hours. In another aspect of the disclosed zeolite support, the reaction mixture is aged about 2 hours to about 100 hours at about 10° C. to about 80° C. before crystallization.

One aspect, the disclosed catalyst comprises:
a) about 0.1% to about 1% by weight of an active metal;
b) about 40% to about 90% by weight of an organosilicon microporous zeolite; and
c) about 10% to about 60% by weight of a binder.

The active metal is one or more elements selected from the group consisting of palladium, ruthenium, platinum, nickel, copper, and cobalt. In one embodiment the active metal is palladium or ruthenium or a combination thereof.

In one embodiment of this aspect the catalyst comprises about 20% to about 40% by weight of a binder. In one iteration of this embodiment of this aspect the binder is selected from the group consisting of aluminum oxide, titanium oxide, zinc oxide, and zirconium oxide.

Another aspect of the disclosed catalysts, comprises:
a) about 0.1% to about 5% by weight of an active metal;
b) about 1% to about 20% by weight of a rare earth metal oxide;
c) about 40% to about 90% by weight of an organosilicon microporous zeolite; and
d) about 10% to about 60% by weight of a binder.

The active metal is one or more elements selected from the group consisting of palladium, ruthenium, platinum, rhodium, or iridium. In one embodiment of this aspect the active metal is palladium or ruthenium or a combination thereof. In another embodiment of this aspect the catalyst comprises about 0.1 to about 3% by weight of an active metal.

In one embodiment of this aspect the rare earth metal oxide is selected from the group consisting of cerium oxide, lanthanum oxide, and zirconium oxide. In another embodiment of this aspect the catalyst comprises about 5% to about 15% by weight of a rare earth metal oxide.

In one embodiment of this aspect the catalyst comprises about 50% to about 70% by weight of an organosilicon microporous zeolite.

In one embodiment of this aspect the catalyst comprises about 20% to about 40% by weight of a binder. In one iteration of this embodiment of this aspect the binder is selected from the group consisting of aluminum oxide, titanium oxide, zinc oxide, and zirconium oxide.

In a further aspect the disclosed catalysts, comprise:
a) about 0.05% to about 5% by weight of an active metal;
b) about 0.05% to about 10% by weight of co-active component;
c) about 40% to about 85% by weight of an organosilicon microporous zeolite; and
d) about 10% to about 50% by weight of a binder.

The active metal is one or more elements selected from the group consisting of palladium, ruthenium, platinum, rhodium, or iridium. In one embodiment of this aspect the active metal is palladium or ruthenium or a combination thereof. In another embodiment of this aspect the catalyst comprises about 0.1 to about 3% by weight of an active metal.

In one embodiment of the disclosed catalyst the co-active component is selected from the group consisting of vanadium chromium, manganese, iron, cobalt, nickel, copper, and zinc. In another embodiment of this aspect the catalyst comprises about 0.1 to about 8% by weight of co-active active component.

In one embodiment of this aspect the catalyst comprises about 50% to about 80% by weight of an organosilicon microporous zeolite.

In one embodiment of this aspect the catalyst comprises about 15% to about 45% by weight of an organosilicon microporous zeolite. In one iteration of this embodiment of this aspect the binder is selected from the group consisting of aluminum oxide, titanium oxide, zinc oxide, and zirconium oxide.

The disclosed organosilicon microporous zeolite support has the formula:

(1/n)Al₂O₃:SiO₂:(m/n)R

wherein n is an index from 5 to 250; m is an index from 0.01 to 50; R is C₁-C₈ alkyl or phenyl.

In one non-limiting embodiment, the index n is from 10 to 100 and the index m is from 0.05 to 20.

In one non-limiting example, R is chosen from methyl or ethyl.

The disclosed organosilicon microporous zeolite of this aspect is characterized by an ²⁹Si NMR spectrum having resonance peaks between −80 and +50 ppm; and an X-ray diffraction pattern with maximum d-spacing of 12.4±0.2, 11.0±0.3, 9.3±0.3, 6.8±0.2, 6.1±0.2, 5.5±0.2, 4.4±0.2, 4.0±0.2 and 3.4±0.1 angstroms.

Sources of inorganic silicon source silica sol, solid silica, silica gel, silicon acetate, diatomaceous earth, or sodium silicate (water glass). In one embodiment of the organosilicon source is at least one selected from halosilane, silazane or alkoxysilane. In another embodiment the source of halosilane is selected from the group consisting of trimethylchlorosilane, dimethyldichlorosilane, triethylchloridesilane, diethyldichlorosilane, dimethylchlorobromosilane, dimethylethylchlorosilane, dimethylbutylchlorosilane, dimethylphenylchlorosilane, dimethylisopropylchlorosilane. In one non-limiting embodiment, two silanes are used, the silanes selected from the group consisting of methyl tert-butylchlorosilane, dimethyloctadecyl-chlorosilane, methylphenylvinylchlorosilane, vinyltrichlorosilane, and diphenyldichlorosilane.

In one non-limiting embodiment the source of aluminum source is selected from the group consisting of sodium aluminate, sodium metaaluminate, aluminum sulfate, aluminum nitrate, aluminum chloride, aluminum hydroxide, aluminum oxide, kaolin, and montmorillonite.

An inorganic base is used in the formation of the disclosed zeolites. Non-limiting examples of which include lithium hydroxide, sodium hydroxide, potassium hydroxide, rubidium hydroxide or cesium hydroxide. In one embodiment, organic bases are used to form the catalyst zeolite. Non-limiting examples include ethylenediamine, hexamethylene-diamine, cyclohexylamine, hexamethyleneimine, heptamethyleneimine, pyridine, hexahydro-pyridine, butylamine, hexylamine, octylamine, quinamine. In one iteration of the process dodecylamine, hexadecylamine or octadecylamine is used.

### SYNTHESIS OF ZEOLITES

### Example 1

The following is a non-limiting example of the procedure for forming the disclosed organosilicon microporous zeolite. Further iterations of the reagents are disclosed herein below.

Sodium aluminate (6.1 g) (Al₂O₃; 42% by weight) was dissolved in water (288 mL) and sodium hydroxide (1.0 g) and the mixture stirred until the reagents had fully dissolved. Hexahydropyridine (34.0g) was added and mixing was continued. Solid silica (60.0 g) and trimethylchlorosilane (5.5 g) was then added. Table 1 lists the molar ratio of the reactants:

TABLE 1

| Reactants | ratio |
|---|---|
| SiO₂/Al₂O₃ | 40 |
| NaOH/SiO₂ | 0.025 |
| Trimethylchlorosilane/SiO₂ | 0.05 |
| Hexahydropyridine/SiO₂ | 0.5 |
| Water/SiO₂ | 16 |

The reaction mixture is stirred until homogeneous then transferred to a stainless-steel reactor and the solution crystalized at 135° C. for 50 hours under agitation. The contents of the reactor are then filtered, washed, and dried. Chemical analysis provides the final product composition as 42:1 SiO₂:Al₂O₃. ²⁹Si NMR shows a resonance at 15.1 ppm.

### Example 2—Synthesis of Ru/MP

The following is a non-limiting example of the procedure for forming the disclosed catalysts. The zeolite formed in Example 1 (65 g) and alumina (35 g) are combined and mixed until homogeneous. Ruthenium (III) chloride, RuCl₃, (100 g as a 5% by weight solution in dilute nitric acid for a final yield of 0.3% by weight ruthenium) is added and the mixture kneaded then extruded into strips. After drying, the strips are roasted at 550° C. for 5 hours, then exchanged ×5 with 1M ammonium nitrate, filtered and dried at 120° C. for 12 hours. The dried strips are then calcined at 480° C. for 6 hours.

### Example 3—Synthesis of Pd/MP

The zeolite formed in Example 1 (65 g) and alumina (35 g) are combined and mixed until homogeneous. Palladium nitrate, Pd(NO₃)₂, (100 g as a 5% by weight solution in dilute nitric acid for a final yield of 0.3% by weight palladium) is added and the mixture kneaded then extruded into strips. After drying, the strips are roasted at 550° C. for 5 hours, then exchanged ×5 with 1M ammonium nitrate, filtered and dried at 120° C. for 12 hours. The dried strips are then calcined at 480° C. for 6 hours.

### Example 4—Synthesis of Pt/MP

The zeolite formed in Example 1 (65 g) and alumina (35 g) are combined and mixed until homogeneous. Platinum (II) chlorate, Pt(ClO₃)₂, (100 g as a 5% by weight solution in dilute nitric acid for a final yield of 0.3% by weight platinum) is added and the mixture kneaded then extruded into strips. After drying, the strips are roasted at 550° C. for 5 hours, then exchanged ×5 with 1M ammonium nitrate, filtered and dried at 120° C. for 12 hours. The dried strips are then calcined at 480° C. for 6 hours.

### Example 5

To a suspension of alumina (3.0 g) in water (450 mL) is added sodium hydroxide (16 g) and the solution stirred until the alumina is dissolved. Hexamethyleneimine (34.7 g) is added and the solution stirred. Solid silica (60 g) and dimethyl diethyloxysilane (5.9 g) is added and the reaction stirred. Table 2 lists the molar ratio of the reactants:

TABLE 2

| Reactants | ratio |
|---|---|
| SiO₂/Al₂O₃ | 30 |
| NaOH/SiO₂ | 0.2 |
| dimethyldiethoxysilane/SiO₂ | 0.04 |
| hexamethyleneimine/SiO₂ | 0.35 |
| Water/SiO₂ | 25 |

The reaction mixture is stirred until homogeneous then transferred to a stainless-steel reactor and the solution crystalized at 145° C. for 70 hours under agitation. The contents of the reactor were then filtered, washed, and dried. Chemical analysis provides the final product composition as 30:1 SiO₂:Al₂O₃. ²⁹Si NMR shows a resonance at −18.9 ppm.

### Example 6—Synthesis of Ru/MP

The zeolite formed in Example 5 (65 g) and alumina (35 g) are combined and mixed until homogeneous. Ruthenium (III) chloride, RuCl₃, (100 g as a 5% by weight solution in dilute nitric acid for a final yield of 0.3% by weight ruthenium) is added and the mixture kneaded then extruded into strips. After drying, the strips are roasted at 550° C. for 5 hours, then exchanged ×5 with 1M ammonium nitrate, filtered and dried at 120° C. for 12 hours. The dried strips are then calcined at 480° C. for 6 hours.

### Example 7—Synthesis of Pd/MP

The zeolite formed in Example 5 (65 g) and alumina (35 g) are combined and mixed until homogeneous. Palladium nitrate, Pd(NO₃)₂, (100 g as a 5% by weight solution in dilute nitric acid for a final yield of 0.3% by weight palladium) is added and the mixture kneaded then extruded into strips. After drying, the strips are roasted at 550° C. for 5 hours, then exchanged ×5 with 1M ammonium nitrate, filtered and dried at 120° C. for 12 hours. The dried strips are then calcined at 480° C. for 6 hours.

### Example 8

Sodium aluminate (3.5 g) (Al₂O₃; 42% by weight) was dissolved in water (540 mL) and sodium hydroxide (8.0 g) and the mixture stirred until the reagents had fully dissolved. Hexamethyleneimine (30.0g) was added and mixing was continued. Solid silica (60.0 g) and hexamethyl disiloxane (8.0 g) was then added. Table 3 lists the molar ratio of the reactants:

TABLE 3

| Reactants | ratio |
|---|---|
| SiO₂/Al₂O₃ | 70 |
| NaOH/SiO₂ | 0.2 |
| hexamethoxydisiloxane/SiO₂ | 0.05 |
| hexamethyleneimine/SiO₂ | 0.3 |
| Water/SiO₂ | 30 |

The reaction mixture is stirred until homogeneous then transferred to a stainless-steel reactor and the solution crystalized at 135° C. for 35 hours under agitation. The contents of the reactor were then filtered, washed, and dried. Chemical analysis provides the final product composition as 68.5:1 SiO₂:Al₂O₃. ²⁹Si NMR shows a resonance at 16.8 ppm.

### Example 9—Synthesis of Ru/MP

The following is a non-limiting example of the procedure for forming the disclosed catalysts. The zeolite formed in Example 8 (65 g) and alumina (35 g) are combined and mixed until homogeneous. Ruthenium (III) chloride, RuCl₃, (100 g as a 5% by weight solution in dilute nitric acid for a final yield of 0.3% by weight ruthenium) is added and the mixture kneaded then extruded into strips. After drying, the strips are roasted at 550° C. for 5 hours, then exchanged ×5 with 1M ammonium nitrate, filtered and dried at 120° C. for 12 hours. The dried strips are then calcined at 480° C. for 6 hours.

### Example 10—Synthesis of Pd/MP

The zeolite formed in Example 8 (65 g) and alumina (35 g) are combined and mixed until homogeneous. Palladium nitrate, Pd(NO₃)₂, (100 g as a 5% by weight solution in dilute nitric acid for a final yield of 0.3% by weight palladium) is added and the mixture kneaded then extruded into strips. After drying, the strips are roasted at 550° C. for 5 hours, then exchanged ×5 with 1M ammonium nitrate, filtered and dried at 120° C. for 12 hours. The dried strips are then calcined at 480° C. for 6 hours.

### Example 11

To a suspension of sodium aluminate (8.0 g) in water (360 mL) is added sodium hydroxide (4.0 g) and the solution stirred until the alumina is dissolved. Hexahydropyridine (34.0 g) was added and the solution stirred. Silica sol (150 g, silica content 45% by weight) and dimethyldichlorosilane (3.9 g) was added and the reaction stirred. Table 4 lists the molar ratio of the reactants:

TABLE 4

| Reactants | ratio |
|---|---|
| SiO₂/Al₂O₃ | 30 |
| NaOH/SiO₂ | 0.05 |
| dimethyldiethoxysilane/SiO₂ | 0.03 |
| hexamethyleneimine/SiO₂ | 0.4 |
| Water/SiO₂ | 20 |

The reaction mixture is stirred until homogeneous then transferred to a stainless-steel reactor and the solution crystalized at 150° C. for 55 hours under agitation. The contents of the reactor were then filtered, washed, and dried. Chemical analysis provides the final product composition as 28.6:1 SiO₂:Al₂O₃. ²⁹Si NMR shows resonance peaks at 5.7 ppm and −16.4 ppm.

### Example 12—Synthesis of Ru/MP

The following is a non-limiting example of the procedure for forming the disclosed catalysts. The zeolite formed in Example 11 (65 g) and alumina (35 g) are combined and mixed until homogeneous. Ruthenium (III) chloride, RuCl₃, (100 g as a 5% by weight solution in dilute nitric acid for a final yield of 0.3% by weight ruthenium) is added and the mixture kneaded then extruded into strips. After drying, the strips are roasted at 550° C. for 5 hours, then exchanged ×5 with 1M ammonium nitrate, filtered and dried at 120° C. for 12 hours. The dried strips are then calcined at 480° C. for 6 hours.

### Example 13—Synthesis of Pd/MP

The zeolite formed in Example 11 (65 g) and alumina (35 g) are combined and mixed until homogeneous. Palladium nitrate, Pd(NO₃)₂, (100 g as a 5% by weight solution in dilute nitric acid for a final yield of 0.3% by weight palladium) is added and the mixture kneaded then extruded into strips. After drying, the strips are roasted at 550° C. for 5 hours, then exchanged ×5 with 1M ammonium nitrate, filtered and dried at 120° C. for 012 hours. The dried strips are then calcined at 480° C. for 6 hours.

### Example 14

To a suspension of sodium aluminate (2.4 g) in water (900 mL) is added sodium hydroxide (4.0 g) and the solution stirred until the alumina is dissolved. Hexamethylene-imine (20.0 g) was added, and the solution stirred. Solid silica (60 g) and divinyldichloro-silane (48.5 g) was added, and the reaction stirred. Table 5 lists the molar ratio of the reactants:

TABLE 5

| Reactants | ratio |
|---|---|
| SiO₂/Al₂O₃ | 100 |
| NaOH/SiO₂ | 1.0 |
| divinyldichlorosilane/SiO₂ | 0.3 |
| hexamethyleneimine/SiO₂ | 0.2 |
| Water/SiO₂ | 50 |

The reaction mixture is stirred until homogeneous then transferred to a stainless-steel reactor and the solution crystalized at 135° C. for 35 hours under agitation. The contents of the reactor were then filtered, washed, and dried. Chemical analysis provides the final product composition as 105.3:1 SiO₂:Al₂O₃. ²⁹Si NMR shows resonance peaks at 4.2 ppm and −17.5 ppm.

### Example 15—Synthesis of Ru/MP

The following is a non-limiting example of the procedure for forming the disclosed catalysts. The zeolite formed in Example 14 (65 g) and alumina (35 g) are combined and mixed until homogeneous. Ruthenium (III) chloride, RuCl₃, (100 g as a 5% by weight solution in dilute nitric acid for a final yield of 0.3% by weight ruthenium) is added and the mixture kneaded then extruded into strips. After drying, the strips are roasted at 480° C. for 6 hours, then exchanged ×5 with 1M ammonium nitrate, filtered and dried at 120° C. for 12 hours. The dried strips are then calcined at 480° C. for 6 hours.

### Example 16—Synthesis of Pd/MP

The zeolite formed in Example 14 (65 g) and alumina (35 g) are combined and mixed until homogeneous. Palladium nitrate, Pd(NO₃)₂, (100 g as a 5% by weight solution in dilute nitric acid for a final yield of 0.3% by weight palladium) is added and the mixture kneaded then extruded into strips. After drying, the strips are roasted at 550° C. for 5 hours, then exchanged ×5 with 1M ammonium nitrate, filtered and dried at 120° C. for 12 hours. The dried strips are then calcined at 480° C. for 6 hours.

### Example 17

To a suspension of sodium aluminate (16.1 g) in water (540 mL) is added sodium hydroxide (2.0 g) and the solution stirred until the alumina is dissolved. Hexamethylene-imine (30.0 g) was added, and the solution stirred. Solid silica (60 g) and disilazane (3.2 g) was added, and the reaction stirred. Table 6 lists the molar ratio of the reactants:

TABLE 6

| Reactants | ratio |
|---|---|
| SiO₂/Al₂O₃ | 15 |
| NaOH/SiO₂ | 0.05 |
| dimethyldiethoxysilane/SiO₂ | 0.04 |
| hexamethyleneimine/SiO₂ | 0.3 |
| Water/SiO₂ | 30 |

The reaction mixture is stirred until homogeneous then transferred to a stainless-steel reactor and the solution crystalized at 145° C. for 38 hours under agitation. The contents of the reactor were then filtered, washed, and dried. Chemical analysis provides the final product composition as 17.5:1 SiO₂:Al₂O₃. ²⁹Si NMR shows a resonance peak at 14.8 ppm.

### Example 18—Synthesis of Ru/MP

The following is a non-limiting example of the procedure for forming the disclosed catalysts. The zeolite formed in Example 17 (65 g) and alumina (35 g) are combined and mixed until homogeneous. Ruthenium (III) chloride, RuCl₃, (100 g as a 5% by weight solution in dilute nitric acid for a final yield of 0.3% by weight ruthenium) is added and the mixture kneaded then extruded into strips. After drying, the strips are roasted at 550° C. for 5 hours, then exchanged ×5 with 1M ammonium nitrate, filtered and dried at 120° C. for 12 hours. The dried strips are then calcined at 480° C. for 6 hours.

### Example 19—Synthesis of Pd/MP

The zeolite formed in Example 17 (65 g) and alumina (35 g) are combined and mixed until homogeneous. Palladium nitrate, Pd(NO₃)₂, (100 g as a 5% by weight solution in dilute nitric acid for a final yield of 0.3% by weight palladium) is added and the mixture kneaded then extruded into strips. After drying, the strips are roasted at 550° C. for 5 hours, then exchanged ×5 with 1M ammonium nitrate, filtered and dried at 120° C. for 12 hours. The dried strips are then calcined at 480° C. for 6 hours.

### Example 20

To a suspension of sodium aluminate (1.6 g) in water (720 mL) is added sodium hydroxide (24.0 g) and the solution stirred until the alumina is dissolved. Hexamethylene-imine (30.0 g) was added, and the solution stirred. Solid silica (60 g) and trimethoxyphenyl-silane (19.8 g) was added, and the reaction stirred. Table 7 lists the molar ratio of the reactants:

TABLE 7

| Reactants | ratio |
|---|---|
| SiO₂/Al₂O₃ | 150 |
| NaOH/SiO₂ | 0.6 |
| divinyldichlorosilane/SiO₂ | 0.1 |
| hexamethyleneimine/SiO₂ | 0.5 |
| Water/SiO₂ | 40 |

The reaction mixture is stirred until homogeneous then transferred to a stainless-steel reactor and the solution crystalized at 135° C. for 35 hours under agitation. The contents of the reactor were then filtered, washed, and dried. Chemical analysis provides the final product composition as 142:1 SiO₂:Al₂O₃. ²⁹Si NMR shows a resonance peak at 17.1 ppm.

### Example 21—Synthesis of Ru/MP

The following is a non-limiting example of the procedure for forming the disclosed catalysts. The zeolite formed in Example 20 (65 g) and alumina (35 g) are combined and mixed until homogeneous. Ruthenium (III) chloride, RuCl₃, (100 g as a 5% by weight solution in dilute nitric acid for a final yield of 0.3% by weight ruthenium) is added and the mixture kneaded then extruded into strips. After drying, the strips are roasted at 550° C. for 5 hours, then exchanged ×5 with 1M ammonium nitrate, filtered and dried at 120° C. for 12 hours. The dried strips are then calcined at 480° C. for 6 hours.

### Example 22—Synthesis of Pd/MP

The zeolite formed in Example 21 (65 g) and alumina (35 g) are combined and mixed until homogeneous. Palladium nitrate, Pd(NO₃)₂, (100 g as a 5% by weight solution in dilute nitric acid for a final yield of 0.3% by weight palladium) is added and the mixture kneaded then extruded into strips. After drying, the strips are roasted at 550° C. for 5 hours, then exchanged ×5 with 1M ammonium nitrate, filtered and dried at 120° C. for 12 hours. The dried strips are then calcined at 480° C. for 6 hours.

### Example 23—Synthesis of Ru/Ni/MP

A solution of 40 mL of ruthenium chloride and nickel nitrate was prepared wherein the content of Ru in the solution is 0.25 g, the content of Ni(NO₃)₂ is 0.23 g, the molar ratio of Ru to Ni is 2. The Ru/Ni solution was then sprayed onto a sample of the organosilicon microporous zeolite powder prepared according to Example 5 (2.50 g), while continuously stirring the zeolite powder. After air-drying at normal temperature for 10 hours, the mixture was dried at 120° C. for 10 hours and ground into powder. Alumina (17.5 g) together with a 5% by weight solution of nitric acid was added for kneading and extruded to form strips of 1.6×2 mm. After drying, the mixture was calcined at 550° C. for 5 hours and then exchanged 5 times with 1M ammonium nitrate, filtered and dried. The composition was dried at 120° C. for 12 hours, roasting at 480° C. for 6 hours to yield the desired catalyst. The content of the final product was found to be Ru is 0.5%, Ni is 0.2%, binder is 34.8%, and organosilicon microporous zeolite is 64.6%.

### Example 24—Synthesis of Ru/Fe/MP

A solution of 40 mL of ruthenium chloride and ferric nitrate was prepared wherein the content of Ru in the solution is 0.25 g, the content of Fe(NO₃)₃ is 0.3 g, the molar ratio of Ru to Fe is 2:3. The Ru/Fe solution was then sprayed onto a sample of the organosilicon microporous zeolite powder prepared according to Example 5 (2.50 g), while continuously stirring the zeolite powder. After air-drying at normal temperature for 10 hours, the mixture was dried at 120° C. for 10 hours and ground into powder. Alumina (17.5 g) together with a 5% by weight solution of nitric acid was added for kneading and extruded to form strips of 1.6×2 mm. After drying, the mixture was calcined at 550° C. for 5 hours and then exchanged 5 times with 1M ammonium nitrate, filtered and dried. The composition was dried at 120° C. for 12 hours, roasting at 480° C. for 6 hours to yield the desired catalyst. The content of the final product was found to be Ru is 0.5%, Fe is 0.2%, binder is 34.8%, and organosilicon microporous zeolite is 64.5%.

### Example 25—Synthesis of Ru/Cu/MP

A solution of 40 mL of ruthenium chloride and cupric nitrate was prepared wherein the content of Ru in the solution is 0.25 g, the content of Cu(NO₃)₂ is 0.23 g, the molar ratio of Ru to Cu is 2. The Ru/Cu solution was then sprayed onto a sample of the organosilicon microporous zeolite powder prepared according to Example 5 (2.50 g), while continuously stirring the zeolite powder. After air-drying at normal temperature for 10 hours, the mixture was dried at 120° C. for 10 hours and ground into powder. Alumina (17.5 g) together with a 5% by weight solution of nitric acid was added for kneading and extruded to form strips of 1.6×2 mm. After drying, the mixture was calcined at 550° C. for 5 hours and then exchanged 5 times with 1M ammonium nitrate, filtered and dried. The composition was dried at 120° C. for 12 hours, roasting at 480° C. for 6 hours to yield the desired catalyst. The content of the final product was found to be Ru is 0.5%, Cu is 0.2%, binder is 34.8%, and organosilicon microporous zeolite is 64.5%.

### Example 26—Synthesis of Pd/Ni/MP

A solution of 40 mL of palladium nitrate and nickel nitrate was prepared wherein the content of Pd in the solution is 0.25 g, the content of Ni(NO₃)₂ is 0.22 g, the molar ratio of Pd to Ni is 2. The Pd/Ni solution was then sprayed onto a sample of the organosilicon microporous zeolite powder prepared according to Example 5 (2.50 g), while continuously stifling the zeolite powder. After air-drying at normal temperature for 10 hours, the mixture was dried at 120° C. for hours and ground into powder. Alumina (17.5 g) together with a 5% by weight solution of nitric acid was added for kneading and extruded to form strips of 1.6×2 mm. After drying, the mixture was calcined at 550° C. for 5 hours and then exchanged 5 times with 1M ammonium nitrate, filtered and dried. The composition was dried at 120° C. for 12 hours, roasting at 480° C. for 6 hours to yield the desired catalyst. The content of the final product was found to be Pd is Ni is 0.2%, binder is 34.8%, and organosilicon microporous zeolite is 64.6%.

### Example 27—Synthesis of Pd/Fe/MP

A solution of 40 mL of palladium nitrate and iron nitrate was prepared wherein the content of Pd in the solution is 0.25 g, the content of Fe(NO₃)₃ is 0.28 g, the molar ratio of Pd to Fe is 2:3. The Pd/Fe solution was then sprayed onto a sample of the organosilicon microporous zeolite powder prepared according to Example 5 (2.50 g), while continuously stirring the zeolite powder. After air-drying at normal temperature for 10 hours, the mixture was dried at 120° C. for hours and ground into powder. Alumina (17.5 g) together with a 5% by weight solution of nitric acid was added for kneading and extruded to form strips of 1.6×2 mm. After drying, the mixture was calcined at 550° C. for 5 hours and then exchanged 5 times with 1M ammonium nitrate, filtered and dried. The composition was dried at 120° C. for 12 hours, roasting at 480° C. for 6 hours to yield the desired catalyst. The content of the final product was found to be Pd is Fe is 0.2%, binder is 34.8%, and organosilicon microporous zeolite is 64.5%.

### Example 28—Synthesis of Pd/Cu/MP

A solution of 40 mL of palladium nitrate and iron nitrate was prepared wherein the content of Pd in the solution is 0.25 g, the content of Cu(NO₃)₂ is 0.22 g, the molar ratio of Pd to Cu is 2. The Pd/Cu solution was then sprayed onto a sample of the organosilicon microporous zeolite powder prepared according to Example 5 (2.50 g), while continuously stirring the zeolite powder. After air-drying at normal temperature for 10 hours, the mixture was dried at 120° C. for hours and ground into powder. Alumina (17.5 g) together with a 5% by weight solution of nitric acid was added for kneading and extruded to form strips of 1.6×2 mm. After drying, the mixture was calcined at 550° C. for 5 hours and then exchanged 5 times with 1M ammonium nitrate, filtered and dried. The composition was dried at 120° C. for 12 hours, roasting at 480° C. for 6 hours to yield the desired catalyst. The content of the final product was found to be Pd is Ni is 0.2%, binder is 34.8%, and organosilicon microporous zeolite is 64.5%.

### Example 29—Synthesis of Ru/CeO₂/MP

A solution of 40 mL of ruthenium chloride and cerium nitrate was prepared wherein the content of Ru in the solution is 0.25 g, the content of Ce(NO₃)₃ is 16.13 g, the molar ratio of Ru to Ce is 1:20. The Pd/Ce solution was then sprayed onto a sample of the organosilicon microporous zeolite powder prepared according to Example 5 (32.50 g), while continuously stirring the zeolite powder. After air-drying at normal temperature for 10 hours, the mixture was dried at 120° C. for 10 hours and ground into powder. Alumina (8.98 g) together with a 5% by weight solution of nitric acid was added for kneading and extruded to form strips of 1.6×2 mm. After drying, the mixture was calcined at 550° C. for 5 hours and then exchanged 5 times with 1M ammonium nitrate, filtered and dried. The composition was dried at 120° C. for 12 hours, roasting at 480° C. for 6 hours to yield the desired catalyst. The content of the final product was found to be Ru is 0.5%, CeO2 is 17.0%, binder is 18%, and organosilicon microporous zeolite is 65.0%.

### Example 30—Synthesis of Ru/ZrO₂/MP

A solution of 40 mL of ruthenium chloride and zirconium nitrate was prepared wherein the content of Ru in the solution is 0.25 g, the content of Zr(NO₃)₄ is 16.78 g, the molar ratio of Ru to Zr is 1:20. The Ru/Zr solution was then sprayed onto a sample of the organosilicon microporous zeolite powder prepared according to Example 5 (32.50 g), while continuously stirring the zeolite powder. After air-drying at normal temperature for 10 hours, the mixture was dried at room temperature for 10 hours and ground into powder. Alumina (11.4 g) together with a 5% by weight solution of nitric acid was added for kneading and extruded to form strips of 1.6×2 mm. After drying, the mixture was calcined at 55 0° C. for 5 hours and then exchanged 5 times with 1M ammonium nitrate, filtered and dried. The composition was dried at 120° C. for 12 hours, roasting at 480° C. for 6 hours to yield the desired catalyst. The content of the final product was found to be Ru is 0.5%, ZrO₂ is 12.2%, binder is 22.8%, and organosilicon microporous zeolite is 65.0%.

### Example 31—Synthesis of Ru/La₂O₃/MP

A solution of 40 mL of ruthenium chloride and lanthanum nitrate was prepared wherein the content of Ru in the solution is 0.25 g, the content of La(NO₃)₃ is 16.07 g, the molar ratio of Ru to La is 1:20. The Ru/La solution was then sprayed onto a sample of the organosilicon microporous zeolite powder prepared according to Example 5 (32.50 g), while continuously stirring the zeolite powder. After air-drying at normal temperature for 10 hours, the mixture was dried at room temperature for 10 hours and ground into powder. Alumina (9.43 g) together with a 5% by weight solution of nitric acid was added for kneading and extruded to form strips of 1.6×2 mm. After drying, the mixture was calcined at 550° C. for 5 hours and then exchanged 5 times with 1M ammonium nitrate, filtered and dried. The composition was dried at 120° C. for 12 hours, roasting at 480° C. for 6 hours to yield the desired catalyst. The content of the final product was found to be Ru is 0.5%, La₂O₃ is 16.1%, binder is 18.9%, and organosilicon microporous zeolite is 65.0%.

### Example 32—Synthesis of Pd/CeO₂/MP

A solution of 40 mL of palladium nitrate and cerium nitrate was prepared wherein the content of Pd in the solution is 0.25 g, the content of Ce is 16.13 g, the molar ratio of Pd to Ce is 1:20. The Pd/Ce solution was then sprayed onto a sample of the organosilicon microporous zeolite powder prepared according to Example 5 (32.50 g), while continuously stifling the zeolite powder. After air-drying at normal temperature for 10 hours, the mixture was dried at 120° C. for 10 hours and ground into powder. Alumina (8.98 g) together with a 5% by weight solution of nitric acid was added for kneading and extruded to form strips of 1.6×2 mm. After drying, the mixture was calcined at 550° C. for 5 hours and then exchanged 5 times with 1M ammonium nitrate, filtered and dried. The composition was dried at 120° C. for 12 hours, roasting at 480° C. for 6 hours to yield the desired catalyst. The content of the final product was found to be Pd is 0.5%, CeO₂ is 17.0%, binder is 18%, and organosilicon microporous zeolite is 65.0%.

### Example 33—Synthesis of Pd/ZrO₂/MP

A solution of 40 mL of palladium nitrate and zirconium nitrate was prepared wherein the content of Pd in the solution is 0.25 g, the content of Zr is 16.78 g, the molar ratio of Pd to Zr is 1:20. The Pd/Zr solution was then sprayed onto a sample of the organosilicon microporous zeolite powder prepared according to Example 5 (32.50 g), while continuously stirring the zeolite powder. After air-drying at normal temperature for 10 hours, the mixture was dried at 120° C. for 10 hours and ground into powder. Alumina (11.4 g) together with a 5% by weight solution of nitric acid was added for kneading and extruded to form strips of 1.6×2 mm. After drying, the mixture was calcined at 550° C. for 5 hours and then exchanged 5 times with 1M ammonium nitrate, filtered and dried. The composition was dried at 120° C. for 12 hours, roasting at 480° C. for 6 hours to yield the desired catalyst. The content of the final product was found to be Pd is 0.5%, ZrO₂ is 12.2%, binder is 22.8%, and organosilicon microporous zeolite is 65.0%.

### Example 34—Synthesis of Pd/La₂O₃/MP

A solution of 40 mL of palladium nitrate and lanthanum nitrate was prepared wherein the content of Ru in the solution is 0.25 g, the content of La(NO₃)₃ is 16.07 g, the molar ratio of Pd to La is 1:20. The Ru/La solution was then sprayed onto a sample of the organosilicon microporous zeolite powder prepared according to Example 5 (32.50 g), while continuously stirring the zeolite powder. After air-drying at normal temperature for 10 hours, the mixture was dried at room temperature for 10 hours and ground into powder. Alumina (9.43 g) together with a 5% by weight solution of nitric acid was added for kneading and extruded to form strips of 1.6×2 mm. After drying, the mixture was calcined at 550° C. for 5 hours and then exchanged 5 times with 1M ammonium nitrate, filtered and dried. The composition was dried at 120° C. for 12 hours, roasting at 480° C. for 6 hours to yield the desired catalyst. The content of the final product was found to be Pd is 0.5%, La₂O₃ is 16.1%, binder is 18.9%, and organosilicon microporous zeolite is 65.0%.

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

### Illustrative Embodiments

P1. A unit operation comprising: introducing hydrogen gas into a heart-cut reformate stream, directing the heart-cut reformate stream into a catalytic reaction zone comprising a catalyst; and heating the heart-cut reformate stream in the presence of the catalyst to form a product stream; wherein the product stream comprises a commodity fraction and bottoms containing diesel range aromatics capable of being hydrotreated to produce a high cetane diesel fraction.

P2. The unit operation according to paragraph P1 wherein the heart-cut reformate stream comprises about 5% to about 75% by weight of benzene and the unit operation converts at least 40% of the benzene in the heart-cut reformate stream.

P3. The unit operation according to any one of paragraphs P1 or P2 wherein the catalytic reaction zone is heated to a temperature of about 100° C. to about 250° C.

P4. The unit operation according to any one of paragraphs P1 to P3 wherein the hydrogen gas is introduced at a pressure of about 50 psig to about 500 psig.

P5. The unit operation according to any one of paragraphs P1 to P4 wherein the heart-cut reformate stream contains a benzene, toluene, xylene (BTX) stream.

P6. The unit operation according to any one of paragraphs P1 to P6 wherein the product stream comprises a naphtha fraction, the commodity fraction, and a diesel fraction.

P7. The unit operation according to paragraph P6 wherein the naphtha fraction comprises alkyl substituted or unsubstituted C₆-C₈ alkyl hydrocarbon and isomers thereof having a formula CₙH₂ₙ wherein the index n is about 6 to about 8.

P8. The unit operation according to any one of paragraphs P6 to P7 wherein the diesel fraction comprises dicyclohexylbenzene, dicyclohexylcyclohexane, and isomers thereof.

P9. The unit operation according to any one of paragraphs P1 or P8 wherein the commodity fraction comprises cyclohexylbenzene, bicyclohexyl, and isomers thereof.

P10. The unit operation according to any one of paragraphs P1 to P9 wherein the commodity fraction and a pre-diesel fraction are combined to form a diesel fraction.

P11. The unit operation according to any one of paragraphs P1 to P8 wherein any single stream or combined stream can be further hydrotreated to improve diesel properties such as diesel cetane number and/or density.

P12. The unit operation according to any one of paragraphs P1 to P11 wherein the catalyst comprises: a) about 0.1 wt % to about 10 wt % of an active metal; b) about 40 wt % to about 90 wt % of an organosilicon microporous zeolite; and c) about 10 wt % to about 60 wt % of a binder; wherein the active metal is palladium, ruthenium, platinum, nickel, copper, and/or cobalt; the binder is aluminum oxide, titanium oxide, zinc oxide, and/or zirconium oxide.

P13. The unit operation according to paragraph P12 wherein the catalyst comprises ruthenium.

P14. The unit operation according to any one of paragraphs P12 to P13 wherein the catalyst comprises palladium.

P15. The unit operation according to any one of paragraphs P12 to P14 wherein the catalyst comprises platinum.

P16. The unit operation according to any one of paragraphs P1 to P11 wherein the catalyst comprises: a) about 0.1 wt % to about 10 wt % of an active metal; b) about 1 wt % to about 20 wt % of a rare earth metal oxide; c) about 40 wt % to about 90 wt % of an organosilicon microporous zeolite; and d) about 10 wt % to about 60 wt % of a binder

P17. The unit operation according to paragraph P16 wherein the active metal is ruthenium.

P18. The unit operation according to paragraph P16 wherein the active metal is palladium.

P19. The unit operation according to any one of paragraphs P16 to P18 wherein the rare earth metal oxide is cerium oxide.

P20. The unit operation according to any one of paragraphs P16 to P18 wherein the rare earth metal oxide is zirconium oxide.

P21. The unit operation according to any one of paragraphs P16 to P18 wherein the rare earth metal oxide is lanthanum oxide.

P22. The unit operation according to any one of paragraphs P1 to P11 wherein the catalyst comprises: a) about 0.05 wt % to about 10 wt % of an active metal; b) about 0.05 wt % to about 10 wt % of a co-active component; c) about 40 wt % to about 85 wt % of an organosilicon microporous zeolite; and d) about 10 wt % to about 50 wt % of a binder.

P23. The unit operation according to paragraph P22 wherein the active metal is ruthenium.

P24. The unit operation according to paragraph P22 wherein the active metal is palladium.

P25. The unit operation according to any one of paragraphs P22 to P24 wherein the co-active component is nickel, iron, and/or copper.

P26. The unit operation according to any one of paragraphs P12 to P25 wherein the organosilicon microporous zeolite has a formula: (1/n)Al₂O₃:SiO₂:(m/n)R wherein n is an index from 5 to 250; m is an index from 0.01 to 50; and R is C₁-C₈ alkyl or phenyl.

P27. The unit operation according to any one of paragraphs P1 to P26 wherein the heart-cut reformate stream is received from a reformate splitter.

P28. The unit operation according to any one of paragraphs P1 to P27 wherein the product stream is delivered to a separation unit.

P29. A unit operation comprising: introducing hydrogen gas into a heart-cut reformate stream, directing the heart-cut reformate stream into a catalytic reaction zone comprising a catalyst; and heating the heart-cut reformate stream in the presence of the catalyst to form a product stream; wherein the product stream comprises a commodity fraction and bottoms containing diesel range aromatics capable of being hydrotreated to produce a high cetane diesel fraction.

P30. The unit operation according to paragraph P29 wherein the heart-cut reformate stream comprises about 5% to about 75% by weight of benzene and the unit operation converts at least 40% of the benzene in the heart-cut reformate stream.

P31. The unit operation according to paragraph P29 wherein the catalytic reaction zone is heated to a temperature of about 100° C. to about 250° C.

P32. The unit operation according to paragraph P29 wherein the hydrogen gas is introduced at a pressure of about 50 psig to about 500 psig.

P33. The unit operation according to paragraph P29 wherein the heart-cut reformate stream contains a benzene, toluene, xylene (BTX) stream.

P34. The unit operation according to paragraph P29 wherein the product stream comprises a naphtha fraction, the commodity fraction, and a diesel fraction.

P35. The unit operation according to paragraph P34 wherein the naphtha fraction comprises alkyl substituted or unsubstituted C₆-C₈ alkyl hydrocarbon and isomers thereof having a formula CₙH₂ₙ wherein the index n is about 6 to about 8.

P36. The unit operation according to paragraph P34 wherein the diesel fraction comprises dicyclohexylbenzene, dicyclohexylcyclohexane, and isomers thereof.

P37. The unit operation according to paragraph P29 wherein the commodity fraction comprises cyclohexylbenzene, bicyclohexyl, and isomers thereof.

P38. The unit operation according to paragraph P29 wherein the commodity fraction and a pre-diesel fraction are combined to form a diesel fraction.

P39. The unit operation according to paragraph P29 wherein any single stream or combined stream can be further hydrotreated to improve diesel properties such as diesel cetane number or density.

P40. The unit operation according to paragraph P29 wherein the heart-cut reformate stream is received from a reformate splitter.

P41. The unit operation according to paragraph P29 wherein the product stream is delivered to a separation unit.

P42. The unit operation according to paragraph P29 wherein the catalyst comprises: a) about 0.1 wt % to about 10 wt % of an active metal; b) about 40 wt % to about 90 wt % of an organosilicon microporous zeolite; and c) about 10 wt % to about 60 wt % of a binder; wherein the active metal is palladium, ruthenium, platinum, nickel, copper, and/or cobalt; wherein the binder is aluminum oxide, titanium oxide, zinc oxide, and/or zirconium oxide.

P43. The unit operation according to paragraph P42 wherein the catalyst comprises ruthenium.

P44. The unit operation according to paragraph P42 wherein the catalyst comprises palladium.

P45. The unit operation according to paragraph P42 wherein the catalyst comprises platinum.

P46. The unit operation according to paragraph P29 wherein the catalyst comprises: a) about 0.1 wt % to about 10 wt % of an active metal; b) about 1 wt % to about 20 wt % of a rare earth metal oxide; c) about 40 wt % to about 90 wt % of an organosilicon microporous zeolite; and d) about 10 wt % to about 60 wt % of a binder.

P47. The unit operation according to paragraph P46 wherein the active metal is ruthenium.

P48. The unit operation according to paragraph P47 wherein the rare earth metal oxide is cerium oxide.

P49. The unit operation according to paragraph P47 wherein the rare earth metal oxide is zirconium oxide.

P50. The unit operation according to paragraph P47 wherein the rare earth metal oxide is lanthanum oxide.

P51. The unit operation according to paragraph P46 wherein the active metal is palladium.

P52. The unit operation according to paragraph P51 wherein the rare earth metal oxide is cerium oxide.

P53. The unit operation according to paragraph P51 wherein the rare earth metal oxide is zirconium oxide.

P54. The unit operation according to paragraph P51 wherein the rare earth metal oxide is lanthanum oxide.

P55. The unit operation according to paragraph P29 wherein the catalyst comprises: a) about 0.05 wt % to about 10 wt % of an active metal; b) about 0.05 wt % to about 10 wt % of a co-active component; c) about 40 wt % to about 85 wt % of an organosilicon microporous zeolite; and d) about 10 wt % to about 50 wt % of a binder.

P56. The unit operation according to paragraph P55 wherein the active metal is ruthenium.

P57. The unit operation according to paragraph P56 wherein the active metal is ruthenium and the co-active component is nickel.

P58. The unit operation according to paragraph P56 wherein the active metal is ruthenium and the co-active component is iron.

P59. The unit operation according to paragraph P56 wherein the active metal is ruthenium and the co-active component is copper.

P60. The unit operation according to paragraph P55 wherein the active metal is palladium.

P61. The unit operation according to paragraph P60 wherein the active metal is palladium and the co-active component is nickel.

P62. The unit operation according to paragraph P60 wherein the active metal is palladium and the co-active component is iron.

P63. The unit operation according to paragraph P60 wherein the active metal is palladium and the co-active component is copper.

P64. The unit operation according to paragraph P55 wherein the organosilicon microporous zeolite has a formula: (1/n)Al₂O₃:SiO₂:(m/n)R wherein n is an index from 5 to 250; m is an index from 0.01 to 50; and R is C₁-C₈ alkyl or phenyl.

What is claimed is:

1. A unit operation comprising:
introducing hydrogen gas into a heart-cut reformate stream, directing the heart-cut reformate stream into a catalytic reaction zone comprising a catalyst;
and heating the heart-cut reformate stream in the presence of the catalyst to form a product stream;
wherein the product stream comprises a commodity fraction and bottoms containing diesel range aromatics capable of being hydrotreated to produce a high cetane diesel fraction.

2. The unit operation according to claim 1 wherein the heart-cut reformate stream comprises about 5% to about 75% by weight of benzene and the unit operation converts at least 40% of the benzene in the heart-cut reformate stream.

3. The unit operation according to claim 1 wherein the catalytic reaction zone is heated to a temperature of about 100° C. to about 250° C.

4. The unit operation according to claim 1 wherein the hydrogen gas is introduced at a pressure of about 50 psig to about 500 psig.

5. The unit operation according to claim 1 wherein the heart-cut reformate stream contains a benzene, toluene, xylene (BTX) stream.

6. The unit operation according to claim 1 wherein the product stream comprises a naphtha fraction, the commodity fraction, and a diesel fraction.

7. The unit operation according to claim 6 wherein the naphtha fraction comprises alkyl substituted or unsubstituted C₆-C₈ alkyl hydrocarbon and isomers thereof having a formula CₙH₂ₙ wherein the index n is about 6 to about 8.

8. The unit operation according to claim 6 wherein the diesel fraction comprises dicyclohexylbenzene, dicyclohexylcyclohexane, and isomers thereof.

9. The unit operation according to claim 1 wherein the commodity fraction comprises cyclohexylbenzene, bicyclohexyl, and isomers thereof.

10. The unit operation according to claim 1 wherein the commodity fraction and a pre-diesel fraction are combined to form a diesel fraction.

11. The unit operation according to claim 1 wherein any single stream or combined stream can be further hydrotreated to improve diesel properties such as diesel cetane number and/or density.

12. The unit operation according to claim 1 wherein the catalyst comprises:
a) about 0.1 wt % to about 10 wt % of an active metal;
b) about 40 wt % to about 90 wt % of an organosilicon microporous zeolite; and
c) about 10 wt % to about 60 wt % of a binder;
wherein the active metal is palladium, ruthenium, platinum, nickel, copper, and/or cobalt;
wherein the binder is aluminum oxide, titanium oxide, zinc oxide, and/or zirconium oxide.

13. The unit operation according to claim 12 wherein the catalyst comprises ruthenium, palladium, or platinum.

14. The unit operation according to claim 1 wherein the catalyst comprises:
a) about 0.1 wt % to about 10 wt % of an active metal;
b) about 1 wt % to about 20 wt % of a rare earth metal oxide;
c) about 40 wt % to about 90 wt % of an organosilicon microporous zeolite; and
d) about 10 wt % to about 60 wt % of a binder.

15. The unit operation according to claim 14 wherein the active metal is ruthenium and/or palladium.

16. The unit operation according to claim 15 wherein the rare earth metal oxide is cerium oxide, zirconium oxide, and/or lanthanum oxide.

17. The unit operation according to claim 1 wherein the catalyst comprises:
a) about 0.05 wt % to about 10 wt % of an active metal;
b) about 0.05 wt % to about 10 wt % of a co-active component;
c) about 40 wt % to about 85 wt % of an organosilicon microporous zeolite; and
d) about 10 wt % to about 50 wt % of a binder.

18. The unit operation according to claim 17 wherein the active metal is ruthenium and/or palladium.

19. The unit operation according to claim 18 wherein the co-active component is nickel, iron, or copper.

20. The unit operation according to claim 17 wherein the organosilicon microporous zeolite has a formula:
(1/n)Al₂O₃:SiO₂:(m/n)R
wherein n is an index from 5 to 250; m is an index from 0.01 to 50; and R is C₁-C₈ alkyl or phenyl.
